(19) United States
(12) Patent Application Publication
(10) Pub. No.: US 2024/0009138 A1
(43) Pub. Date: Jan. 11, 2024

(54) EDIBLE GRAPHIC DRUG DELIVERY SYSTEM AND METHOD OF MANUFACTURE

(71) Applicant: Bryan Daniel HAYNES, Alameda, CA (US)

(72) Inventor: Bryan Daniel HAYNES, Alameda, CA (US)

(21) Appl. No.: 18/355,325

(22) Filed: Jul. 19, 2023

Related U.S. Application Data
(63) Continuation of application No. 17/374,986, filed on Jul. 13, 2021, now abandoned.
(60) Provisional application No. 63/054,591, filed on Jul. 21, 2020.

(51) Int. Cl. A61K 9/70; A61K 47/36; A61K 9/00

(52) U.S. Cl. CPC A61K 9/7007; A61K 47/36; A61K 9/0056

(57) ABSTRACT

A dissolvable film delivery system formed by printing one or more layers of inks formulated to deliver specific ingredients and convey graphic imagery and textual information. The dissolvable film is printed to convey a graphic image or textual message using a single printed layer. The dissolvable film may further be printed using multiple layers of inks that incorporate different ingredients into the overall graphic design to achieve a desired effect from incorporated layers. The dissolvable graphic is further printed in layers to achieve a method of encapsulation allowing for a sequential delivery of ingredients comprising flavors and dosage of active ingredients.

### RELATED APPLICATION DATA

The present application claims priority under 35 U.S.C. § 119 to U.S. Provisional Application Ser. No. 63/054,591, naming Haynes as the inventor, filed Jul. 21, 2020, and entitled EDIBLE GRAPHICS EXTRACT DELIVERY SYSTEM AND METHOD OF MANUFACTURE, which is incorporated herein by reference in its entirety and for all purposes.

### FIELD OF THE INVENTION

The present invention relates generally to oral medicine delivery, and more specifically sublingual and buccal dissolvable films infused with active ingredients capable of being fabricated by printing thin layers of edible inks which contain flavoring and colors specific to each layer to fabricate a colorful graphic display infused with flavoring and active ingredients that dissolves in the mouth.

### BACKGROUND OF THE INVENTION

Conventional sublingual dissolvable strips have remained relatively unchanged since their creation. Primarily used as a breath freshening device conventional sublingual dissolvable strips are manufactured as a single layer, single flavor, single color, single medicine and are limited to a square or rectangular shape that does not offer a means of graphical display. More recently, an array of additives to the sublingual strip have advanced the technology providing for medicinal use beyond freshening breath.

With the addition of medicinal ingredients to the sublingual film industry, there has been little development or technological advancement to the sublingual strips appearance. Without any textual or graphical markings indicating dosage, ingredients, or warnings, all sublingual strips look identical to one another. Two potential sublingual strip identification methods such as printing onto or die cutting shapes out of sublingual film may be employed, however the application of such methods would require additional equipment beyond the formation of the sublingual film. Unfortunately, the inclusion of either method would incur significant costs or waste.

### SUMMARY OF THE INVENTION

Generally, according to the present invention sublingual delivery devices can be printed in a variety of shapes, colors, flavors, and dosages forming a dissolvable graphic image that is easily manufactured to incorporate graphic imagery and textual information associated to logo, brand, manufacturer, dosage ingredients, instructions, and effects of corresponding medicine.

The oral delivery dissolvable film system according to the present invention is well suited to provide graphical information incorporated into individual doses whereas conventional film drug delivery strips are manufactured through a process that does not lend itself to the incorporation of graphical information without the use of a secondary process not normally associated to the fabrication of drug delivery strips.

One embodiment of the present invention is that multiple medicines or ingredients may be included into a single sublingual device by printing sequential layers of infused inks enabling the desired effects of medicines included within a single sublingual device without the cross contamination and distortion of individual formulas that may occur by including multiple medicines within a single liquid slurry.

In another configuration of the present invention a sublingual device may incorporate isolated regions of active ingredients by means of encapsulation through printing a sequence of layers to form a printed pocket wherein the printed encapsulation perimeters extend beyond the perimeter of the active ingredient.

In another embodiment of the present invention individual screen-printed layers are combined in sequence to incorporate color and separate stencils to form a sublingual device where all layers form a combined graphic image.

In yet another embodiment of the present invention a safety barrier of non-infused layers may be printed to form a non-medicinal encapsulation wherein the first and last or outermost layers form a non-medicinal barrier to the infused layers of the sublingual device.

### BRIEF DESCRIPTION OF THE DRAWINGS

The assembly of the present invention has other objects and features of advantage which will be more readily apparent from the following description of the best mode of carrying out the invention and the appended claims, when taken in conjunction with the accompanying drawing, in which:

FIG. 1 is a cross section of a printed sublingual delivery device within an array of printed sublingual delivery devices constructed in accordance with the present invention.

FIG. 2 is a top view of a printed sublingual delivery device within an array of printed sublingual delivery devices of FIG. 1.

FIG. 3 is a side view cutaway of a printed three-layer sublingual delivery device deposited on a substrate.

FIG. 4 is a top view of a printed sublingual delivery device that illustrates text and graphics deposited onto a previous printed layer.

FIG. 5 is a cross section of a printed sublingual delivery device deposited on a substrate that enables the release of active ingredients or flavors in sequence that illustrates two layers of encapsulation by printing four layers encapsulating one final layer.

FIG. 6 is a cross section of a printed sublingual delivery device printed on a substrate that illustrates two layers side by side for simultaneous release of flavors or active ingredients encapsulated by two printed layers.

FIG. 7 is a top view of a printed sublingual delivery device that illustrates the difference in perimeter size required for encapsulation.

FIG. 8 is a cross section of a printed sublingual delivery device that illustrates the application of layers deposited to fabricate a three-layer encapsulated sublingual delivery device deposited on a substrate.

FIG. 9 is a cross section of a printed sublingual delivery device that illustrates the sequential release of encapsulated flavors and active ingredients and deposited on a substrate.

FIGS. 10A-10D is a sequence of fragmentary, top plan views of the printed sublingual delivery device of FIG. 1, illustrating the layered fabrication.

FIG. 11 is a top view of an array of printed sublingual delivery devices that illustrates a printed sublingual delivery device and the use of printed perforations to establish dosage.

### DETAILED DESCRIPTION

While the present invention will be described with reference to a few specific embodiments, the description is illustrative of the invention and is not to be construed as limiting the invention. Various modifications to the present invention can be made to the preferred embodiments by those skilled in the art without departing from the true spirit and scope of the invention as defined by the appended claims. It will be noted here that for a better understanding, like components are designated by like reference numerals throughout the various figures.

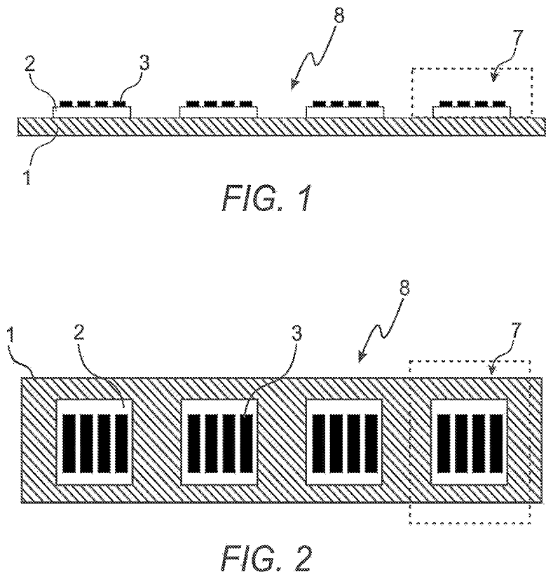

The scope of the invention in this embodiment demonstrates the spirit of the base invention as shown in FIG. 1, where a sublingual delivery device, generally designated 7 is formed by the application of first print layer 2 consisting of an edible ink where colors, flavoring and active ingredients are infused and deposited onto a substrate 1, wherein first print layer 2, once cured and peeled off substrate 1 to be further consumed as a sublingual delivery device 7.

While this configuration of the sublingual delivery device 7 is fabricated using various conventional techniques, this substantially flat sublingual delivery device 7 is particularly suitable for printed fabrication. That is, most if not all of the subsequent material layers, atop one another, can be applied using printing techniques. Such techniques include, but are not limited to, offset printing, flexo-graphic printing, roto gravure, ink jet deposition, screen-printing and pad printing.

Referring now to FIGS. 1 and 2, a printed graphic sublingual film delivery device 7, within an array of printed sublingual strips 8 is deposited by printing first print layer 2 which may or may not include graphics and text to represent the overall scope of the invention by printing a specified shape onto a flexible substrate 1, which additionally may be followed by a second print layer 3 which may be provided as a purely graphical indicia in order to convey the perceived visual subject matter as demonstrated in FIG. 4 of a specific sublingual strip device 7.

A significant advantage of printing a sublingual device 7 is print manufacturing eliminates the need for die-cutting to achieve a specified shape and therefore eliminating the waste associated to die-cutting where the space in between the specified shapes would be discarded thereby losing active ingredients as waste.

The viscosity of the ink is selected to enable optimal print fabrication, while at the same time being sufficiently viscous to allow the infused active ingredients to be uniformly suspended within the ink.

Another advantage of screen printing a sublingual delivery device 7 is that depositing layers by printing allows for multiple prints for depositing a specified amount of ink to achieve a desired thickness of ink. The composition and forward build printing sequence of the sublingual delivery device 7 will now be described using FIGS. 1, 2, and 4. It will be appreciated, however, that the thickness of each layer shown is not proportional, and merely shown as being equal for illustrative purposes.

In one embodiment, the sublingual delivery device 7 is formed by depositing first print layer 2 which may also be referred to as base layer, which may include multiple prints for depositing a specified amount or achieve a desired thickness. First print layer 2 is deposited on substrate 1 which may be described as a transparent sheet or film such as MYLAR Polyester Film, Polyethylene terephthalate (PET) and may or may not be removed from the finished sublingual delivery device 7.

In another embodiment, a second print layer 3 is deposited by printing onto first print layer 2 as best shown in FIGS. 1, 2, and 4, which may be comprised of multiple prints for depositing a specified amount or achieve a desired thickness and may also include text and graphics as shown in FIG. 4. Second print layer 3 may or may not be infused with flavoring or active ingredients and may only serve as a layer that depicts text and imagery.

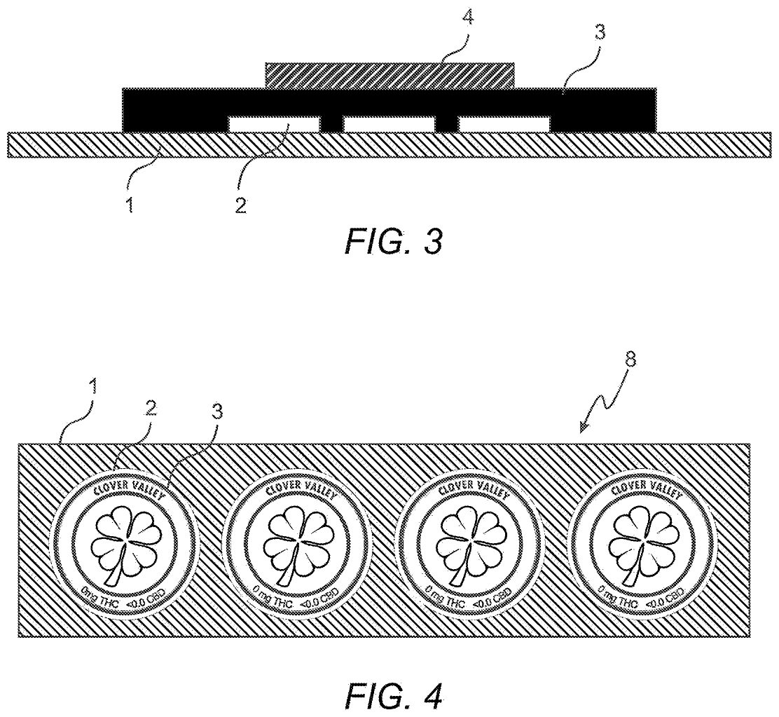

As another embodiment of the present invention, text and imagery may be included in first print layer 2, depicted in FIG. 3, which shows first print layer 2 is obscured or hidden by second print layer 3. Hence, the lack of ability to view the text and graphics of first print layer 2 will need to be deposited in reverse or mirrored in relation to the overall sublingual delivery device 7. Therefore, first print layer 2 will be readable to the viewer on the reverse side or bottom of the sublingual delivery device 7 after it is removed from the substrate 1.

In another embodiment of the present invention, a sublingual delivery device 7 may be peeled off substrate 1 for use where substrate 1 represents a release liner intended for ease of packaging. Increased adhesion to substrate 1 may be increased by modifying the adhesion within printed ink of first print layer 2. Increased adhesion may be the only objective of first print layer 2 wherein no color, flavor, or active ingredient is used in the adhesion layer.

As best viewed in FIG. 4, in one embodiment, second print layer 3 conveys a textual and graphic message within a single layer and represents a layer that may or may not be infused with active ingredients, wherein the layer containing active ingredients within the sublingual delivery device 7 may be within first print layer 2.

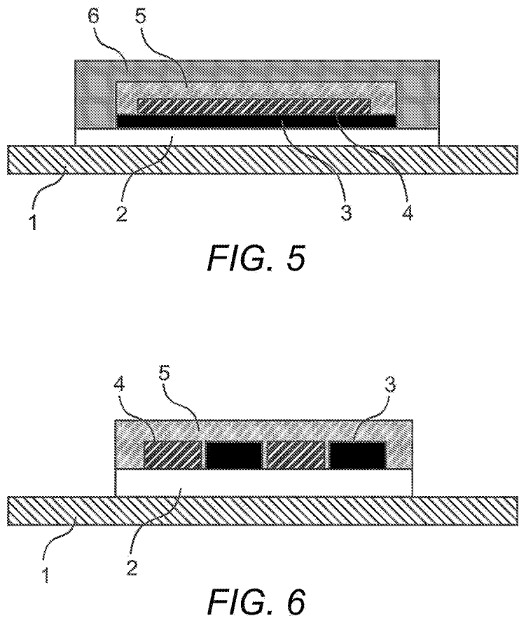

Referring now to FIG. 5, another specific embodiment of a sublingual delivery device 7 is illustrated, in cross section, having a significantly unique embodiment according to the present invention, wherein third print layer 4 is encapsulated by second print layer 3, fourth print layer 5, first print layer 2 and fifth print layer 6. This print construction allows for the sequential release of active ingredients or flavorings where first print layer 2 and fifth print layer 6 share corresponding flavor or active ingredients and second print layer 3 and fourth print layer 5 share corresponding flavor or active ingredients which demonstrates two encapsulating layers that must dissolve before the release of flavor or active ingredients of third print layer 4.

Referring now to FIG. 6, another specific embodiment of a sublingual delivery device 7 which demonstrates the simultaneous release of flavors, active ingredients contained within second print layer 3 and third print layer 4. This is accomplished by means of encapsulation, wherein first print layer 2 represents the first encapsulant layer and fourth print layer 5 represents the second encapsulant layer.

In still another specific embodiment of FIG. 6, encapsulated layers may offer a duration of disintegration or a variable dissolving rate that enables the ability to calculate time between release of flavors and active ingredients in sublingual delivery device 7.

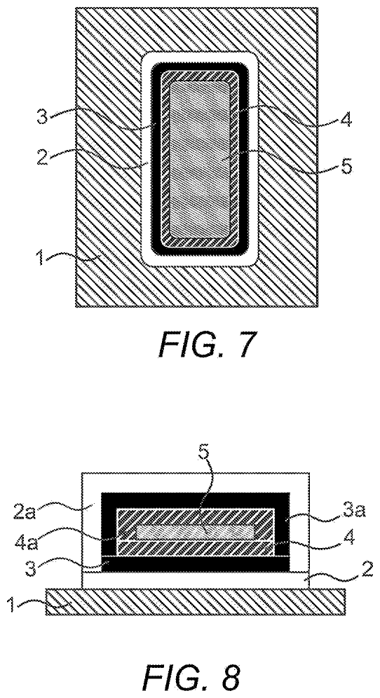

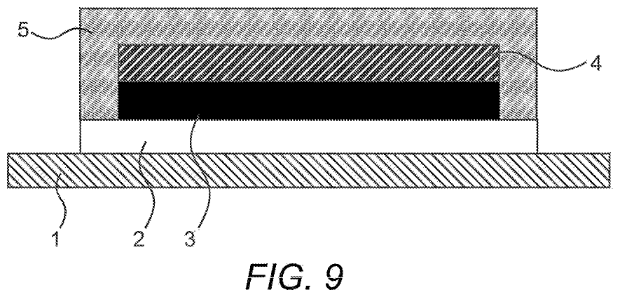

Turning now to FIG. 7, which demonstrates the printing of concentric layers as a method of encapsulation where top view clearly shows defined perimeters of each printed layer of sublingual delivery device 7, wherein first print layer 2 represents the first layer to dissolve in sequence, followed by second print layer 3, third print layer 4, fourth print layer 5 which represents the final layer containing active ingredients or flavoring dissolves. Each printed layer has a corresponding opposing layer as demonstrated in FIG. 8.

FIG. 8 illustrates a side view and completed construction of FIG. 7 by demonstrating additional layers that enable encapsulation and the sequential release of flavoring and active ingredients. This is accomplished by depositing first print layer 2, second print layer 3, third print layer 4, and fourth print layer 5, the innermost print layer of the final construction, followed by what is considered the corresponding opposing layers print layer 4a, print layer 3a, and print layer 2a. In this view the corresponding opposing layers are represented as being of the same composition as its opposing layer. For example, first print layer 2 and eighth print layer 2a are represented as the same material.

Turning now to FIG. 9, is another specific embodiment of a sublingual delivery device 7 which demonstrates yet another method of the sequential release of flavors, active ingredients contained within second print layer 3 and third print layer 4. This is accomplished by means of encapsulation where first print layer 2 represents the first encapsulant layer and fourth print layer 5 represents the second encapsulant layer.

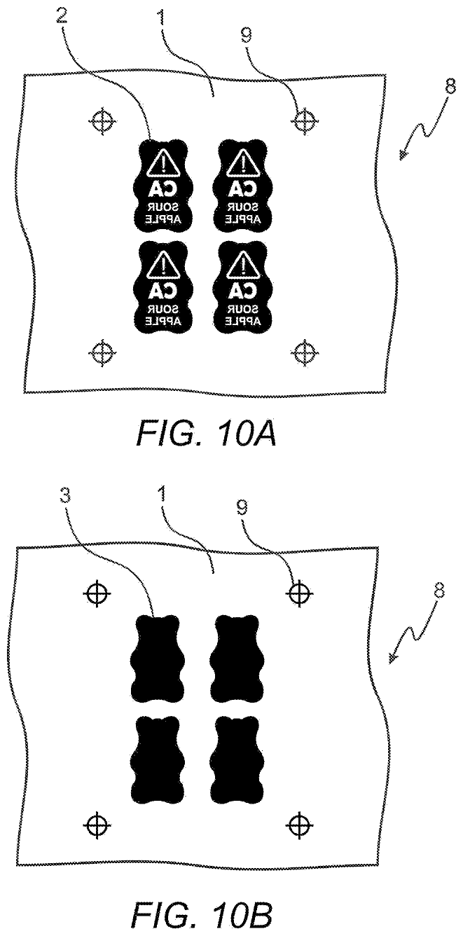

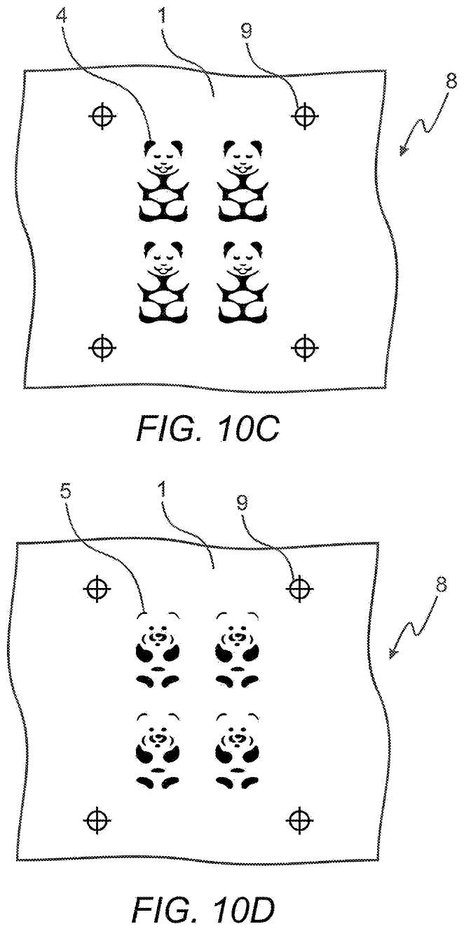

Briefly, the composition and forward build printing sequence of the sublingual delivery device 7 will now be described using FIGS. 10A-10D.

Referring now to FIG. 10A, first print layer 2 is the first layer deposited onto substrate 1 and represents the first of four print layers in sequence required to form a combined graphic image within an array of sublingual delivery devices 8. Here, first print layer 2 incorporates text and graphics printed in reverse in relation to the forward build of the overall sublingual delivery device 7. Alignments or registration marks 9 are incorporated into FIGS. 10A-10D to demonstrate the alignment and registration required for the optimal outcome of the finished sublingual delivery device 7.

FIG. 10B according to the present invention illustrates the second print layer 3 is then disposed atop first print layer 2 (FIG. 10A), which is also referred to as base layer, which includes multiple prints for depositing a specified volume or achieve a desired thickness necessary to achieve a specified amount of flavoring or active ingredients.

FIG. 10C illustrates third print layer 4 is then disposed atop second print layer 3 (FIG. 10B), in the overall image formed by the four sequential prints of FIGS. 10A-10D. Third print layer 4 represents the shadow print of the overall graphic image.

FIG. 10D illustrates fourth print layer 5 is then disposed atop third print layer 4 (FIG. 10C), in the overall image formed by the four sequential prints of FIGS. 10A-10D. Fourth print layer 5 represents the highlight print of the overall graphic image.

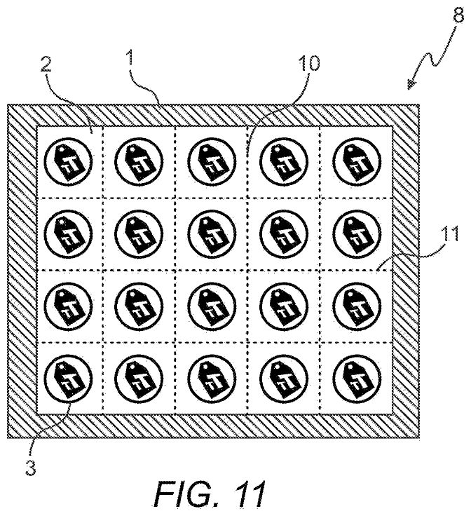

FIG. 11 according to the present invention illustrates the use of printed vertical perforations 10 and printed horizontal perforations 11 that are formed by printing ink and leaving openings within the printed area to form a pattern wherein said openings in such pattern will cause an ease of separation of materials along such patterned line of openings within first printed layer 2 disposed on substrate 1 followed by second print layer 3 to fabricate an array of printed sublingual delivery devices 8 to establish dosage.

Although the present invention has been described in connection with the preferred form of practicing it and modifications thereto, those of ordinary skill in the art will understand that many other modifications can be made thereto within the scope of the claims that follow. Accordingly, it is not intended that the scope of the invention in any way be limited by the above description, but instead be determined entirely by reference to the claims that follow.

1-23. (canceled)

24. A method of preparing a plurality of sublingual delivery devices deposited on a substrate, wherein the sublingual delivery device is peeled off the substrate for human consumption, the method comprising:
(i) screen printing an edible base layer onto the substrate;
(ii) screen printing one or more edible intermediate layers onto the edible base layer, wherein the perimeter of the one or more edible intermediate layers lies within the perimeter of the edible base layer; and
(iii) screen printing an edible top layer onto the edible base layer and the one or more edible intermediate layers, wherein the perimeter of the edible top layer is equal to the perimeter of the edible base layer, such that the edible top layer and the edible base layer together encapsulate the one or more edible intermediate layers,
wherein one or more active ingredients is independently present in the edible base layer, any of the one or more edible intermediate layers, or the edible top layer, or any combination thereof, such that the total amount of each of the one or more active ingredients present in a single sublingual delivery device is sufficient to deliver a predetermined dosage to a subject in need thereof; and
wherein the predetermined dosage of active ingredients is controlled by the thickness or height of edible layers comprising one or more of the active ingredients.

25. The method of claim 24, wherein the substrate is a release liner, and
wherein adhesion of the sublingual delivery device to the substrate is controlled by the adhesion within the printed edible base layer.

26. The method of claim 24, wherein the substrate comprises paper, a wax coated substrate, a coated cardboard, plastic, or a metalized substrate.

27. The method of claim 24, wherein the substrate comprises MYLAR Polyester Film or polyethylene terephthalate (PET).

28. The method of claim 24, wherein a plurality of active ingredients is present in the sublingual delivery device, wherein the plurality of active ingredients is printed into sequential edible layers.

29. The method of claim 24, wherein the edible base layer, one or more of the edible intermediate layers, or any combination thereof, is transparent or translucent.

30. The method of claim 24, wherein the edible base layer, the one or more edible intermediate layers, and the edible top layer comprise printed shapes and patterns that correspond to a template recognized by an inkjet printer.

31. The method of claim 24, further comprising inkjet printing one or more additional edible layers to form patterns comprising indicia, text, or graphic images.

32. The method of claim 31, wherein one or more of the additional edible layers deposited by inkjet printing are positioned between the edible base layer and the one or more edible intermediate layers.

33. A method of preparing a plurality of sublingual delivery devices deposited on a substrate, wherein the sublingual delivery device is peeled off the substrate for human consumption, the method comprising screen printing an edible base layer onto the substrate,
wherein one or more active ingredients is independently present in the edible base layer, such that the total amount of each of the one or more active ingredients present in a single sublingual delivery device is sufficient to deliver a predetermined dosage to a subject in need thereof; and
wherein the predetermined dosage of active ingredients is controlled by the thickness or height of the edible base layer.

34. The method of claim 33, wherein the substrate is a release liner, and
wherein adhesion of the sublingual delivery device to the substrate is controlled by the adhesion within the printed edible base layer.

35. The method of claim 33, wherein the substrate comprises paper, a wax coated substrate, a coated cardboard, plastic, or a metalized substrate.

36. The method of claim 33, wherein the substrate comprises MYLAR Polyester Film or polyethylene terephthalate (PET).

37. The method of claim 33, wherein a plurality of active ingredients is present in the edible base layer.

38. The method of claim 33, wherein the edible base layer is transparent or translucent.

39. The method of claim 33, wherein the edible base layer comprises printed shapes and patterns that correspond to a template recognized by an inkjet printer.

40. The method of claim 33, further comprising inkjet printing one or more additional edible layers to form patterns comprising indicia, text, or graphic images.

41. A sublingual delivery device, comprising:
(i) an edible base layer screen printed onto a substrate;
(ii) one or more edible intermediate layers screen printed onto the edible base layer, wherein the perimeter of the one or more edible intermediate layers lies within the perimeter of the edible base layer; and
(iii) an edible top layer screen printed onto the edible base layer and the one or more edible intermediate layers, wherein the perimeter of the edible top layer is equal to the perimeter of the edible base layer, such that the edible top layer and the edible base layer together encapsulate the one or more edible intermediate layers,
wherein one or more active ingredients is independently present in the edible base layer, any of the one or more edible intermediate layers, or the edible top layer, or any combination thereof, such that the total volume of each of the one or more active ingredient present in a single sublingual delivery device is sufficient to deliver a predetermined dosage to a subject in need thereof;
wherein the predetermined dosage of active ingredients is controlled by the thickness or height of edible layers comprising one or more of the active ingredients; and
wherein the sublingual delivery device is peeled off the substrate for human consumption.

42. The device of claim 41, wherein the substrate is a release liner, and
wherein adhesion of the sublingual delivery device to the substrate is controlled by the adhesion within the printed edible base layer.

43. The device of claim 41, wherein the substrate comprises paper, a wax coated substrate, a coated cardboard, plastic, or a metalized substrate.

44. The device of claim 41, wherein the substrate comprises MYLAR Polyester Film or polyethylene terephthalate (PET).

45. The device of claim 41, wherein a plurality of active ingredients is present in the sublingual delivery device, wherein the plurality of active ingredients is printed into sequential edible layers.

46. The device of claim 41, wherein the edible base layer, one or more of the edible intermediate layers, or any combination thereof, is transparent or translucent.

47. The device of claim 41, wherein the edible base layer, the one or more edible intermediate layers, and the edible top layer comprise printed shapes and patterns that correspond to a template recognized by an inkjet printer.

48. The device of claim 41, further comprising one or more additional edible layers deposited by inkjet printing to form patterns comprising indicia, text, or graphic images.

49. The device of claim 48, wherein one or more of the additional edible layers deposited by inkjet printing are positioned between the edible base layer and the one or more edible intermediate layers.

50. The device of claim 41, comprising:
(i) an edible base layer printed onto a substrate;
(ii) a first edible intermediate layer printed onto the edible base layer, wherein the perimeter of the first intermediate layer lies within the perimeter of the edible base layer;
(iii) a second edible intermediate layer printed onto the first edible intermediate layer, wherein the perimeter of the second edible intermediate layer lies within the perimeter of the first edible intermediate layer;
(iv) a third edible intermediate layer printed onto the second edible intermediate layer and the first edible intermediate layer, wherein the perimeter of the third edible intermediate layer is equal to the perimeter of the first edible intermediate layer; and
(v) an edible top layer printed onto the third intermediate layer and the edible base layer, wherein the perimeter of the edible top layer is equal to the perimeter of the edible base layer,
wherein a plurality of active ingredients is present in the sublingual delivery device, wherein the plurality of active ingredients is printed into sequential edible layers, such that the release of active ingredients occurs in sequence.

51. The device of claim 41, comprising:
(i) an edible base layer printed onto a substrate;
(ii) a first edible intermediate layer printed onto the edible base layer, wherein the perimeter of the first intermediate layer lies within the perimeter of the edible base layer;
(iii) a second edible intermediate layer and a third edible intermediate layer printed onto the first edible intermediate layer, wherein the perimeters of the second edible intermediate layer and the third edible intermediate layer lie within the perimeter of the first edible intermediate layer, and wherein the second intermediate layer and the third edible intermediate layer are deposited onto the first edible intermediate layer; and
(iv) an edible top layer printed onto the second intermediate edible layer, the third intermediate layer, and the edible base layer, wherein the perimeter of the edible top layer is equal to the perimeter of the edible base layer,
wherein a plurality of active ingredients is present in the sublingual delivery device, wherein the plurality of active ingredients is printed into sequential edible layers, such that the release of active ingredients occurs in sequence, with the release of the active ingredients present in the second edible intermediate layer and the third edible intermediate layer occurring simultaneously.

52. The device of claim 41, comprising:
(i) an edible base layer printed onto a substrate;
(ii) a first edible intermediate layer printed onto the edible base layer, wherein the perimeter of the first intermediate layer lies within the perimeter of the edible base layer;
(iii) a second edible intermediate layer printed onto the first edible intermediate layer, wherein the perimeter of the second edible intermediate layer lies within the perimeter of the first edible intermediate layer;
(iv) a third edible intermediate layer printed onto the second edible intermediate layer, wherein the perimeter of the third edible intermediate layer lies within the perimeter of the second edible intermediate layer;
(v) a fourth edible intermediate layer printed onto the third edible intermediate layer and the second edible intermediate layer, wherein the perimeter of the fourth edible intermediate layer is equal to the perimeter of the second edible intermediate layer;
(vi) a fifth edible intermediate layer printed onto the fourth edible intermediate layer and the first edible intermediate layer, wherein the perimeter of the fifth edible intermediate layer is equal to the perimeter of the first edible intermediate layer; and
(vii) an edible top layer printed onto the fifth edible intermediate layer and the edible base layer, wherein the perimeter of the edible top layer is equal to the perimeter of the edible base layer,
wherein a plurality of active ingredients is present in the sublingual delivery device, wherein the plurality of active ingredients is printed into sequential edible layers, such that the release of active ingredients occurs in sequence.

53. The device of claim 41, comprising:
(i) an edible base layer printed onto a substrate;
(ii) a first edible intermediate layer printed onto the edible base layer, wherein the perimeter of the first intermediate layer lies within the perimeter of the edible base layer;
(iii) a second edible intermediate layer printed onto the first edible intermediate layer, wherein the perimeter of the second edible intermediate layer is equal to the perimeter of the first edible intermediate layer; and
(iv) an edible top layer printed onto the second edible intermediate layer and the edible base layer, wherein the perimeter of the edible top layer is equal to the perimeter of the edible base layer,
wherein a plurality of active ingredients is present in the sublingual delivery device, wherein the plurality of active ingredients is printed into sequential edible layers, such that the release of active ingredients occurs in sequence.

54. An array comprising a plurality of sublingual delivery devices of claim 41,
wherein each sublingual delivery device within the array is separated by space on the substrate, wherein the space does not comprise active ingredients; and
wherein each sublingual delivery device in the array has the same specified shape and predetermined dosage of the one or more active ingredients.

55. An array comprising a plurality of sublingual delivery devices of claim 41,
wherein each sublingual delivery device within the array is separated by perforations that allow for the separation of individual sublingual delivery devices;
wherein the perforations are formed by the absence of edible ink material in a pattern of holes; and
wherein each sublingual delivery device in the array has the same specified shape and predetermined dosage of the one or more active ingredients.

56. A sublingual delivery device comprising an edible base layer screen printed onto a substrate,
wherein one or more active ingredients is independently present in the edible base layer, such that the total volume of each of the one or more active ingredients present in a single sublingual delivery device is sufficient to deliver a predetermined dosage to a subject in need thereof;
wherein the predetermined dosage of active ingredients is controlled by the thickness or height of the edible base layer; and
wherein the sublingual delivery device is peeled off the substrate for human consumption.

57. The device of claim 56, wherein the substrate is a release liner, and
wherein adhesion of the sublingual delivery device to the substrate is controlled by the adhesion within the printed edible base layer.

58. The device of claim 56, wherein the substrate comprises paper, a wax coated substrate, a coated cardboard, plastic, or a metalized substrate.

59. The device of claim 56, wherein the substrate comprises MYLAR Polyester Film or polyethylene terephthalate (PET).

60. The device of claim 56, wherein a plurality of active ingredients is present in the edible base layer.

61. The device of claim 56, wherein the edible base layer is transparent or translucent.

62. The device of claim 56, wherein the edible base layer comprises printed shapes and patterns that correspond to a template recognized by an inkjet printer.

63. The device of claim 56, further comprising one or more additional edible layers deposited by inkjet printing to form patterns comprising indicia, text, or graphic images.

64. An array comprising a plurality of sublingual delivery devices of claim 56,
wherein each sublingual delivery device within the array is separated by space on the substrate, wherein the space does not comprise active ingredients; and
wherein each sublingual delivery device in the array has the same specified shape and predetermined dosage of the one or more active ingredients.

65. An array comprising a plurality of sublingual delivery devices of claim 56,
wherein each sublingual delivery device within the array is separated by perforations that allow for the separation of individual sublingual delivery devices;
wherein the perforations are formed by the absence of edible ink material in a pattern of holes; and
wherein each sublingual delivery device in the array has the same specified shape and predetermined dosage of the one or more active ingredients.
